(19) United States
(12) Patent Application Publication
(10) Pub. No.: US 2024/0091183 A1
(43) Pub. Date: Mar. 21, 2024

(54) TREATMENT REGIMENS WITH FIXED DOSES OF TAMIBAROTENE

(71) Applicant: Syros Pharmaceuticals, Inc., Cambridge, MA (US)

(72) Inventors: David A. Roth, Wellesley, MA (US); Michael J. Kelly, West Newton, MA (US); Catherine E. Madigan, Harvard, MA (US); Maria Rosario, Boston, MA (US)

(21) Appl. No.: 18/260,537

(22) PCT Filed: Jan. 7, 2022
(86) PCT No.: PCT/US22/11670

§ 371 Date: Jul. 6, 2023

Related U.S. Application Data
(60) Provisional application No. 63/135,450, filed on Jan. 8, 2021.

(51) Int. Cl. A61K 31/196; A61K 31/635; A61K 31/706; A61K 45/06; A61P 35/02

(52) U.S. Cl. CPC A61K 31/196; A61K 31/635; A61K 31/706; A61K 45/06; A61P 35/02

(57) ABSTRACT

The present invention features, inter alia, methods of treating (a) a patient who has been diagnosed with a hematopoietic cancer or (b) a population of patients who have been diagnosed with a hematopoietic cancer with a fixed dose of tamibarotene or a pharmaceutically acceptable salt thereof. The tamibarotene is administered daily, for a prescribed number of days, at 8-14 (e.g., 12 mg/day) regardless of the patient's weight or body surface area, and may be administered as the sole therapeutic agent or in combination with one or more of the additional therapeutic agents described herein.

### CROSS-REFERENCE TO RELATED APPLICATIONS

This application claims the benefit of the filing date of U.S. provisional application No. 63/135,450, filed Jan. 8, 2021, the contents of which is hereby incorporated by reference in its entirety.

### BACKGROUND

Leukemias, lymphomas, myelomas, and myelodysplastic syndromes (MDS) are types of cancer that can affect the bone marrow, the blood cells, the lymph nodes, and other parts of the lymphatic system. It was estimated that almost 200,000 patients in the United States alone were diagnosed with leukemia, lymphoma, or myeloma in 2021, accounting for about 10 percent of all newly diagnosed cancers.

### SUMMARY

The present invention features, inter alia, methods of treating a patient (e.g., an adolescent or adult human) who has been diagnosed as having a hematopoietic cancer, including any one or more of those described herein, with a fixed dose of tamibarotene (i.e., a dose that is given to patients regardless of their weight, body surface area (BSA) or other indicator of size), alone or in combination with a second therapeutic agent (e.g., a hypomethylating agent such as azacitidine or decitabine) and/or a BCL2 inhibitor (e.g., venetoclax). Where more than two agents are employed, we may refer to a “third” therapeutic agent, a “fourth” therapeutic agent, and so forth, with the understanding that these conventional numerical designations are used as a matter of convenience to refer to distinct agents within a plurality; the designations are not meant to convey the order of administration or otherwise restrict the treatment regimen. For example, where tamibarotene, azacitidine, and venetoclax are employed, azacitidine can be referred to as the second therapeutic agent and venetoclax can be referred to as the third therapeutic agent, or vice versa. Where tamibarotene, azacitidine, venetoclax, and magrolimab are employed, venetoclax may be referred to as the third therapeutic agent and magrolimab may be referred to as the fourth therapeutic agent, or vice versa. Accordingly, in a first aspect, the invention features methods of treating a patient who has a hematopoietic cancer, including any one or more of those described herein, the method comprising a step of administering to the patient about 8-14 mg (e.g., 12 mg) of tamibarotene, or a pharmaceutically acceptable salt thereof, per day. In one embodiment, the patient has a hematopoietic cancer, including any one or more of those described herein, and the method comprises a step of administering to the patient about 12 mg of tamibarotene, or a pharmaceutically acceptable salt thereof, per day. For ease of reading, we may not refer to a pharmaceutically acceptable salt of a given compound (e.g., tamibarotene, azacitidine, decitabine, or venetoclax) at every opportunity. It is to be understood that where a compound is useful, a therapeutically effective and pharmaceutically acceptable salt of that compound may also be used. The daily dose of tamibarotene can be divided into multiple daily administrations (e.g., two, three, or four administrations). For example, a daily dose of about 8-14 mg (e.g., 12 mg) of tamibarotene can be divided into two doses, the first being a first daily dose and the second being a second daily dose (administered, e.g., about 12 hours apart, such as around 8 am and around 8 pm; around 9 am and around 9 pm; and so forth). The first daily dose and the second daily dose (as well as any doses beyond two) can include equal or unequal amounts of tamibarotene or a pharmaceutically acceptable salt thereof. For example, when administered twice per day, each dose can contain about 4, 5, 6 or 7 mg of tamibarotene (providing a total of 8, 10, 12, or 14 mg of tamibarotene to the patient per day). With respect to tableting, the about 8-14 mg (e.g., 12 mg) can be contained in a single tablet or multiple tablets. For example, for a dose of 12 mg/day, a patient could take two tablets, each containing about 3 mg of tamibarotene, in the morning and two tablets at night (i.e., 6 mg in the morning and six at night). Alternatively, for a dose of 12 mg/day, a patient could take three tablets, each containing about 2 mg of tamibarotene, in the morning and three tablets at night (still, 6 mg in the morning and six at night). The same is true should the tablet be altered to another dosage form, for example, a capsule. In case of doubt, these precise dosage amounts and regimens are within the scope of the present invention and can be administered with any more of the additional (second, third, fourth) therapeutic agents described herein. Tamibarotene or the pharmaceutically acceptable salt thereof can be administered orally, and the fixed dose (e.g., the about 8-14 mg (e.g., 12 mg) total daily dose) can be administered in any aspect or embodiment of the invention. For example, a fixed dose of 8-14 mg (e.g., 12 mg) can be administered regardless of the specific hematopoietic cancer being treated, regardless of whether tamibarotene is administered as the sole therapeutic agent for treating the cancer or is administered together with one or more additional therapeutic agents, including any one or more of those set out herein, and regardless of the dosage form (e.g., a tablet or capsule) or schedule (e.g., administered once or twice per day). The hematopoietic cancer can be a leukemia, a lymphoma, multiple myeloma, or myeloid neoplasm with myelodysplasia (e.g., a myelodysplastic syndrome (MDS)). For example, the leukemia can be acute lymphoblastic leukemia (ALL), chronic lymphocytic leukemia (CLL), acute myeloid leukemia (AML), or chronic myeloid leukemia (CML), and the lymphoma can be Hodgkin lymphoma or non-Hodgkin lymphoma. In some embodiments, the AML is non-acute promyelocytic leukemia acute myeloid leukemia (non-APL AML). The terminology used here is consistent with Arber et al. (Blood 127(20):2391-2405, 2016). Where the hematopoietic cancer is a myeloid neoplasm with myelodysplasia, it can be MDS or a subtype thereof (e.g., MDS with single-lineage dysplasia, MDS with multi-lineage dysplasia, MDS with ring sideroblasts and single- or multi-lineage dysplasia, MDS with isolated del(5q), MDS with excess blasts type 1 or type 2; or MDS unclassifiable) or a myelodysplastic-myeloproliferative neoplasm (i.e., a myeloid neoplasm with clinical, laboratory, and/or morphologic features that overlap those of MDS and myeloproliferative neoplasms) or a subtype thereof (e.g., chronic myelomonocytic leukemia (CMML), BCR-ABL1-negative atypical chronic myeloid leukemia, myelodysplastic-myeloproliferative neoplasm with ring sideroblasts and thrombocytosis, or juvenile myelomonocytic leukemia) (see Cazzola, New Engl. J. Med. 383:1358-1374, 2020). In other embodiments, the hematopoietic cancer is hypoplastic MDS or MDS with fibrosis. Where the hematopoietic cancer is an MDS, the MDS can be higher risk MDS (HR-MDS) based on the revised International Prognostic Scoring System (IPSS-R) or the World Health Organization (WHO) Prognostic Scoring System (WPSS). Alternatively, the MDS can be lower risk MDS (LR-MDS) as defined by the IPSS-R or WPSS. In any aspect or embodiment, the methods can include administering a second therapeutic agent to the patient. For example, the second therapeutic agent can be a hypomethylating agent (HMA (e.g., decitabine or azacitidine)) or a BCL2 inhibitor (e.g., venetoclax). Where azacitidine is administered, it can be administered parenterally (e.g., intravenously or subcutaneously) at a dose of about 75 mg/m². In some embodiments, a patient (e.g., an adult human) having a hematopoietic cancer, including any one or more of those disclosed herein, is treated with tamibarotene, administered orally at a dose of about 8-14 mg (e.g., 6 mg twice per day on each of days 8-28 of a 28-day treatment cycle) and with azacitidine, administered intravenously or subcutaneously at a dose of 75 mg/m 2 on each of days 1-7 of the 28-day treatment cycle. The patient may undergo multiple treatment cycles, and the administration of tamibarotene and azacitidine as just described may be accompanied by the administration of a third therapeutic agent (e.g., venetoclax or another BCL2 inhibitor, administered by a protocol known in the art) or by the administration of third and fourth therapeutic agents (e.g., venetoclax, or another BCL2 inhibitor, and obinutuzumab, administered by protocols known in the art).

In any of the aspects and embodiments of the invention (e.g., regardless of the specific hematopoietic cancer to be treated, regardless of whether tamibarotene is administered alone or in combination with a second therapeutic agent, and regardless of the precise dosing regimen (e.g., the precise amount of tamibarotene per tablet or capsule)), the patient may be newly diagnosed (ND) with the cancer and, therefore, treatment naive; may be considered to have a higher risk form of the cancer; may be considered unfit (e.g., unfit for standard or intensive induction chemotherapy); or may be relapsed from or refractory to a prior treatment. In any of the aspects and embodiments of the invention, the diagnosis may include a determination that the patient's cancer cells express one or more biomarkers (e.g., a RARA biomarker as described in U.S. Pat. Nos. 9,845,508 and 9,868,994, each of which is hereby incorporated by reference in its entirety). As is known in the art, the RARA biomarker can comprise elevated expression of a RARA primary RNA transcript (an RNA transcription product from a DNA sequence that includes a coding region of a RARA gene (e.g., at least one exon) and/or a non-coding region of the gene (e.g., an intron or a regulatory region of the gene (e.g., an enhancer or super enhancer that regulates expression of the gene)). Thus, the RARA primary RNA transcript assessed as a biomarker can be an “enhancer RNA” or “eRNA,” a microRNA, a precursor mRNA (“pre-mRNA”) or mature mRNA. As is known in the art, in methods of assessing the level of expression of a RARA primary RNA transcript, one may assess a cDNA that has been synthesized or reverse transcribed from a RARA primary RNA transcript.

Each therapeutic method disclosed herein and described as a “method of treating” a patient or population of patients may also be described in terms of a “use.” For example, the invention encompasses the use of a fixed dose of 10-14 mg (e.g., 12 mg) of tamibarotene for the treatment of a hematopoietic cancer as described herein and the use of such amounts of tamibarotene for the preparation of a medicament for treating a hematopoietic cancer.

### BRIEF DESCRIPTION OF THE DRAWINGS

FIG. 1 is a schematic illustrating the study design described in Example 1.

FIG. 2 is a table highlighting details of the therapeutic agents administered according to the study described in Example 1.

### DETAILED DESCRIPTION

Tamibarotene is a potent and selective agonist of retinoic acid receptor alpha (RARα), approved for the treatment of relapsed/refractory acute promyelocytic leukemia (R/R APL) in Japan (see, e.g., Miwako, Drugs Today 43(8):563-568, 2007, and Tobita et al., Blood 90(3):967-973, 1997). Tamibarotene is being developed in a subset of non-APL acute myeloid leukemia (AML) patients, i.e., those who are RARA-positive, as identified by a clinical trial assay in which RARA expression levels are determined by qPCR. Patients deemed RARA-positive can be identified by this assay, similar assays, or an approved (e.g., FDA-approved) test that detects a super enhancer associated with the RARA gene or high levels of RARA expression in cancer cells (e.g., blasts) within a biological sample (e.g., a blood or bone marrow sample) obtained from the patient. An assay for RARA expression, for example, can be carried out as need by measuring the levels of a RARA primary RNA transcript (e.g., an mRNA transcribed from the RARA gene) in hematopoietic cancer cells obtained from a patient (e.g., by way of a blood or bone marrow sample collection). The cells can be peripheral blood mononuclear cells (PBMCs) and can be determined to be CD34/CD117-positive. RNA transcripts are extracted from the cells and subjected to a measurement technique such as RT-qPCR. RARA expression can be measured relative to one or more control genes (e.g., one or more “housekeeping” genes), and the results can then be assessed to determine whether the transcript levels are elevated (e.g., more than 1.5-fold greater than the expression level of the control gene(s) and/or above a pre-determined threshold level established in the art).

Initial clinical data in RARA-positive R/R AML and R/R HR-MDS patients treated with single agent tamibarotene demonstrated evidence of biological activity with myeloid differentiation and blast reductions (Jurcic et al., American Society of Hematology Annual Meeting, Abstract No. 2633, 2017). In newly diagnosed, unfit AML patients, tamibarotene in combination with azacitidine showed high complete response (CR) rates and a rapid onset of action in RARA-positive patients (de Botton et al., European School of Haematology (ESH) Conference on AML, Abstract No. 16081, 2019). More specifically, in this study, RARA biomarker-positive and RARA biomarker-negative treatment naïve non-APL AML patients were treated with azacitidine (75 mg/m 2 administered intravenously or subcutaneously on days 1-7 of a 28-day treatment cycle) and tamibarotene (at 6 mg/m²/day, by mouth, on days 8-28 of the 28-day treatment cycle). The adverse event (AE) profile of the combination was consistent with what has been previously reported for single agent tamibarotene or single agent azacitidine in treatment of AML/MDS patients. Approximately 30% of AML patients and 30% to 40% of MDS patients are RARA-positive (Jurcic et al., supra; Vigil et al., European School of Haematology (ESH) Conference on AML, Abstract No. 8882, 2018; de Botton et al., supra). Azacitidine remains the standard of care for the treatment of HR-MDS, however there is opportunity to improve upon the clinical outcomes of single-agent azacitidine. In one of the clinical studies we are currently conducting, newly diagnosed, RARA-positive HR-MDS patients are randomized to therapy with tamibarotene plus azacitidine or placebo plus azacitidine (see below).

As indicated, the present method features methods of treating a patient who has a hematopoietic cancer, the methods including a step of administering to the patient about 12 mg of tamibarotene per day. Each daily dose can be divided into two, the first being a first daily dose and the second being a second daily dose. The first daily dose and the second daily dose can each contain about 6 mg of tamibarotene, and the tamibarotene can be administered orally. In any of the methods of the invention, the about 12 mg of tamibarotene is administered regardless of the patient's weight or body surface area. In any of the methods of the invention, the patient can be a child, adolescent, or adult human patient (e.g., an adult human who is elderly and/or deemed unfit for standard induction chemotherapy, and/or relapsed or refractory to a prior treatment). In the methods of the invention, the hematopoietic cancer can be a leukemia (e.g., a leukemia resistant or refractory to treatment with venetoclax or an AML of the M4 or M5 subtype), a lymphoma, multiple myeloma, or myeloid neoplasm with myelodysplasia. More specifically, the leukemia can be acute lymphoblastic leukemia (ALL), chronic lymphocytic leukemia (CLL), acute myeloid leukemia (AML), or chronic myeloid leukemia (CIVIL), and the lymphoma can be Hodgkin lymphoma or non-Hodgkin lymphoma. the AML can be non-acute promyelocytic leukemia acute myeloid leukemia (non-APL AML). The myeloid neoplasm with myelodysplasia can be a myelodysplastic syndrome (MDS) (e.g., higher risk MDS or lower risk MDS). The methods of the invention can include a second step of administering a second therapeutic agent (i.e., an agent distinct from tamibarotene), such as a hypomethylating agent (HMA) or a BCL2 inhibitor. The HMA can be decitabine or azacitidine (or another HMA, including one of those disclosed herein) and the BCL2 inhibitor can be venetoclax. In some embodiments, the HMA is azacitidine. Where azacitidine is administered, it can be administered parenterally at a dose of 75 mg/m². In various embodiments of the present invention, tamibarotene can be administered orally at a dose of 6 mg twice per day on each of days 8-28 of a 28-day treatment cycle and azacitidine can be administered intravenously or subcutaneously at a dose of 75 mg/m 2 on each of days 1-7 of the 28-day treatment cycle. In some embodiments, the methods comprise administering both a second therapeutic agent and a third therapeutic agent, in which case the second therapeutic agent and the third therapeutic agent can be independently selected from an HMA (e.g., azacitidine or decitabine), a BCL2 inhibitor (e.g., venetoclax), low-dose ara-C(LDAC), obinutuzumab, rituximab, a CD47 inhibitor (e.g., magrolimab), and an androgen. In some embodiments, the methods comprise administering a second, a third, and a fourth therapeutic agent independently selected from an HMA (e.g., azacitidine or decitabine), a BCL2 inhibitor (e.g., venetoclax), LDAC, obinutuzumab, rituximab, a CD47 inhibitor (e.g., magrolimab), and an androgen. In embodiments, the second agent is an HMA (e.g., azacitidine or decitabine), the third agent is a BCL2 inhibitor (e.g., venetoclax), and the fourth agent is LDAC; the second agent is an HMA (e.g., azacitidine or decitabine), the third agent is a BCL2 inhibitor (e.g., venetoclax), and the fourth agent is obinutuzumab; the second agent is an HMA (e.g., azacitidine or decitabine), the third agent is a BCL2 inhibitor (e.g., venetoclax), and the fourth agent is rituximab; the second agent is an HMA (e.g., azacitidine or decitabine), the third agent is a BCL2 inhibitor (e.g., venetoclax), and the fourth agent is a CD47 inhibitor (e.g., magrolimab); or the second agent is an HMA (e.g., azacitidine or decitabine), the third agent is a BCL2 inhibitor (e.g., venetoclax), and the fourth agent is an androgen. In embodiments, the methods comprise administering an HMA (e.g., azacitidine or decitabine) as the second agent, a BCL2 inhibitor (e.g., venetoclax) as the third agent, and LDAC as the fourth agent to a patient with AML (e.g., the M4 or M5 subtype of AML); administering an HMA (e.g., azacitidine or decitabine) as the second agent, a BCL2 inhibitor (e.g., venetoclax) as the third agent, and obinutuzumab as the fourth agent to a patient with CLL or SLL; administering an HMA (e.g., azacitidine or decitabine) as the second agent, a BCL2 inhibitor (e.g., venetoclax) as the third agent, and rituximab as the fourth agent to a patient with CLL or SLL, with or without 17p deletion; administering an HMA (e.g., azacitidine or decitabine) as the second agent, a BCL2 inhibitor (e.g., venetoclax) as the third agent, and a CD47 inhibitor (e.g., magrolimab) as the fourth agent to a patient with an MDS; or administering an HMA (e.g., azacitidine or decitabine) as the second agent, a BCL2 inhibitor (e.g., venetoclax) as the third agent, and an androgen as the fourth agent to a patient with persistent anemia.

In another aspect, the invention features methods of treating a population of patients who each have a hematopoietic cancer (e.g., the same type of hematopoietic cancer), the methods including a step of administering to each of the patients about 12 mg of tamibarotene per day regardless of the patient's weight or body surface area. Each daily dose can be divided into two, the first being a first daily dose and the second being a second daily dose. The first daily dose and the second daily dose can each contain about 6 mg of tamibarotene, and the tamibarotene can be administered orally. In any of the methods of treating a population of patients, the patients can children, adolescents, or adult human patients (e.g., adult humans who are elderly and/or deemed unfit for standard induction chemotherapy, and/or relapsed or refractory to a prior treatment). In the methods of treating a population, the hematopoietic cancer can be a leukemia (e.g., a leukemia resistant or refractory to treatment with venetoclax or an AML of the M4 or M5 subtype), a lymphoma, multiple myeloma, or myeloid neoplasm with myelodysplasia. More specifically, the leukemia can be acute lymphoblastic leukemia (ALL), chronic lymphocytic leukemia (CLL), acute myeloid leukemia (AML), or chronic myeloid leukemia (CML), and the lymphoma can be Hodgkin lymphoma or non-Hodgkin lymphoma. the AML can be non-acute promyelocytic leukemia acute myeloid leukemia (non-APL AML). The myeloid neoplasm with myelodysplasia can be a myelodysplastic syndrome (MDS) (e.g., higher risk MDS or lower risk MDS). The methods of the invention can include a second step of administering a second therapeutic agent (i.e., an agent distinct from tamibarotene), such as a hypomethylating agent (HMA) or a BCL2 inhibitor. The HMA can be decitabine or azacitidine (or another HMA, including one of those disclosed herein) and the BCL2 inhibitor can be venetoclax. In some embodiments, the HMA is azacitidine. Where azacitidine is administered, it can be administered parenterally at a dose of 75 mg/m². In various embodiments of the present invention, tamibarotene can be administered orally at a dose of 6 mg twice per day on each of days 8-28 of a 28-day treatment cycle and azacitidine can be administered intravenously or subcutaneously at a dose of 75 mg/m 2 on each of days 1-7 of the 28-day treatment cycle. In some embodiments, the methods comprise administering both a second therapeutic agent and a third therapeutic agent, in which case the second therapeutic agent and the third therapeutic agent can be independently selected from an HMA (e.g., azacitidine or decitabine), a BCL2 inhibitor (e.g., venetoclax), low-dose ara-C(LDAC), obinutuzumab, rituximab, a CD47 inhibitor (e.g., magrolimab), and an androgen. In some embodiments, the methods comprise administering a second, a third, and a fourth therapeutic agent independently selected from an HMA (e.g., azacitidine or decitabine), a BCL2 inhibitor (e.g., venetoclax), LDAC, obinutuzumab, rituximab, a CD47 inhibitor (e.g., magrolimab), and an androgen. In embodiments, the second agent is an HMA (e.g., azacitidine or decitabine), the third agent is a BCL2 inhibitor (e.g., venetoclax), and the fourth agent is LDAC; the second agent is an HMA (e.g., azacitidine or decitabine), the third agent is a BCL2 inhibitor (e.g., venetoclax), and the fourth agent is obinutuzumab; the second agent is an HMA (e.g., azacitidine or decitabine), the third agent is a BCL2 inhibitor (e.g., venetoclax), and the fourth agent is rituximab; the second agent is an HMA (e.g., azacitidine or decitabine), the third agent is a BCL2 inhibitor (e.g., venetoclax), and the fourth agent is a CD47 inhibitor (e.g., magrolimab); or the second agent is an HMA (e.g., azacitidine or decitabine), the third agent is a BCL2 inhibitor (e.g., venetoclax), and the fourth agent is an androgen. In embodiments, the methods comprise administering an HMA (e.g., azacitidine or decitabine) as the second agent, a BCL2 inhibitor (e.g., venetoclax) as the third agent, and LDAC as the fourth agent to a patient with AML (e.g., the M4 or M5 subtype of AML); administering an HMA (e.g., azacitidine or decitabine) as the second agent, a BCL2 inhibitor (e.g., venetoclax) as the third agent, and obinutuzumab as the fourth agent to a patient with CLL or SLL; administering an HMA (e.g., azacitidine or decitabine) as the second agent, a BCL2 inhibitor (e.g., venetoclax) as the third agent, and rituximab as the fourth agent to a patient with CLL or SLL, with or without 17p deletion; administering an HMA (e.g., azacitidine or decitabine) as the second agent, a BCL2 inhibitor (e.g., venetoclax) as the third agent, and a CD47 inhibitor (e.g., magrolimab) as the fourth agent to a patient with an MDS; or administering an HMA (e.g., azacitidine or decitabine) as the second agent, a BCL2 inhibitor (e.g., venetoclax) as the third agent, and an androgen as the fourth agent to a patient with persistent anemia.

The following definitions apply to the compositions, methods, and uses described herein unless the context clearly indicates otherwise, and it is to be understood that the claims may be amended to include language within a definition if needed or desired. Moreover, the definitions apply to linguistic and grammatical variants of the defined terms (e.g., the singular and plural forms of a term), and some linguistic variants are particularly mentioned below (e.g., “administration” and “administering”).

The term “about,” when used in reference to a value, signifies any value or range of values that is within plus-or-minus 5% of the stated value (e.g., within plus-or-minus 1%, 2%, 3%, 4%, or 5% of the stated value). For example, a dose of about 10 mg means any dose as low as 5% less than 10 mg (9.5 mg), any dose as high as 5% more than 10 mg (10.5 mg), and any dose or dosage range therebetween. Where a stated value cannot be exceeded (e.g., 100%), “about” signifies any value or range of values that is up to and including 5% less than the stated value (e.g., a purity of about 100% means 95%-100% pure (e.g., 96%-100% pure, 97%-100% pure etc.)). In the event an instrument or technique measuring a value has a margin of error greater than 5%, a given value will be about the same as a stated value when they are both within the margin of error for that instrument or technique.

The term “administration” and variants thereof, such as “administering,” refer to the administration of a compound described herein (e.g., tamibarotene, an HMA (e.g., azacitidine), a BCL2 inhibitor such as venetoclax, and pharmaceutically acceptable salts thereof) or a pharmaceutical composition containing one or more of such compounds to a subject (e.g., a human patient) or system (e.g., a cell- or tissue-based system that is maintained ex vivo); as a result of the administration, the compound or composition containing the compound is introduced to the subject (e.g., the patient) or system. In addition to active pharmaceutical ingredients, items used as positive controls, negative controls, and placebos, any of which can also be or include a compound, can also be “administered.” One of ordinary skill in the art will be aware of a variety of routes that can, in appropriate circumstances, be utilized for administration to a patient or system. For example, the route of administration can be oral (i.e., by swallowing a pharmaceutical composition) or may be parenteral. More specifically, the route of administration can be bronchial (e.g., by bronchial instillation), by mouth (i.e., oral), dermal (which may be or comprise topical application to the dermis or intradermal, interdermal, or transdermal administration), intragastric or enteral (i.e., directly to the stomach or intestine, respectively), intramedullary, intramuscular, intranasal, intraperitoneal, intrathecal, intratumoral, intravenous (or intra-arterial), intraventricular, by application to or injection into a specific organ (e.g., intrahepatic), mucosal (e.g., buccal, rectal, sublingual, or vaginal), subcutaneous, tracheal (e.g., by intratracheal instillation), or ocular (e.g., topical, subconjunctival, or intravitreal). Administration can involve intermittent dosing (i.e., doses separated by various times) and/or periodic dosing (i.e., doses separated by a common period of time (e.g., every so many hours, daily (e.g., once daily oral dosing), weekly, twice per week, etc.)). In other embodiments, administration may involve continuous dosing (e.g., perfusion) for a selected time (e.g., about 1-2 hours). Therapeutically effective amounts and dosing regimens for the additional (i.e., second, third, and fourth) agents described herein are known in the art, and we expect such amounts and regimens can be used in the present methods, particularly where azacitidine, decitabine, and/or venetoclax are employed.

The term “biological sample” refers to a sample obtained or derived from a biological source of interest (e.g., a tissue or organism (e.g., an animal or human patient) or cell culture). For example, a biological sample can be a sample obtained from an individual (e.g., a patient or an animal model) suffering from a disease (or, in the case of an animal model, a simulation of that disease in a human patient) to be diagnosed and/or treated by the methods of the present disclosure or from an individual serving in the capacity of a reference or control (or whose sample contributes to a reference standard or control population). The biological sample can contain a biological cell (e.g., a cancer cell), tissue or fluid or any combination thereof. For example, a biological sample can be or can include ascites; blood; blood cells; a bodily fluid, any of which may include or exclude cells (e.g., tumor cells (e.g., circulating tumor cells (CTCs) found in at least blood or lymph vessels)); bone marrow or a component thereof (e.g., hematopoietic cells, marrow adipose tissue, or stromal cells); cerebrospinal fluid (CSF); feces; flexural fluid; free-floating nucleic acids (e.g., circulating tumor DNA); gynecological fluids; hair; immune infiltrates; lymph; peritoneal fluid; plasma; saliva; skin or a component part thereof (e.g., a hair follicle); sputum; surgically-obtained specimens; tissue scraped or swabbed from the skin or a mucus membrane (e.g., in the nose, mouth, or vagina); tissue or fine needle biopsy samples; urine; washings or lavages such as a ductal lavage or broncheoalveolar lavage; or other body fluids, tissues, secretions, and/or excretions. A biological sample may include cancer cells (e.g., of a hematopoietic cancer described herein) or immune cells, such as NK cells and/or macrophages, which are found in many tissues and organs, including the spleen and lymph nodes. Samples of, or samples obtained from, a bodily fluid (e.g., blood, CSF, lymph, plasma, or urine) may include tumor cells (e.g., CTCs) and/or free-floating or cell-free nucleic acids. Cells (e.g., cancer cells) within the sample may have been obtained from an individual patient for whom a treatment is intended. Samples used in the form in which they were obtained may be referred to as “primary” samples, and samples that have been further manipulated (e.g., by removing one or more components of the sample) may be referred to as “secondary” or “processed” samples. Such processed samples may contain or be enriched for a particular cell type (e.g., a hematopoietic cancer cell or blast cell), cellular component (e.g., a membrane fraction), or cellular material (e.g., one or more cellular proteins, DNA, or RNA (e.g., mRNA), which may be subjected to amplification).

The term “biologically active” describes an agent (e.g., a compound described herein) that produces an observable biological effect or result in a biological system or model thereof (e.g., in a human, other animal, or a system maintained in cell/tissue culture or in vitro). The “biological activity” of such an agent can manifest upon binding between the agent and a target (e.g., RARA or BCL2)), and it may result in modulation (e.g., induction, enhancement, or inhibition) of a biological pathway, event, or state (e.g., a disease state). For example, the agent can modulate a cellular activity (e.g., stimulation of an immune response or inhibition of homologous recombination repair), time spent in a phase of the cell cycle (which may alter the rate of cellular proliferation), or initiation of apoptosis or activation of another pathway leading to cell death (which may lead to tumor regression). A biological activity and, optionally, its extent, can be assessed using known methods to detect any given immediate or downstream product of the activity or any event associated with the activity (e.g., inhibition of cell growth or tumor regression).

The term “carrier” refers to a diluent, adjuvant, excipient, or other vehicle with which an active pharmaceutical agent (e.g., a compound of the present disclosure, or a pharmaceutically acceptable salt, solvate, stereoisomer, tautomer, or isotopic form thereof) is formulated for administration. The carrier, in the amount and manner incorporated into a pharmaceutical composition, will be non-toxic to the subject and will not destroy the biological activity of the active ingredient (e.g., the compound or other specified form thereof) with which it is formulated. The carrier can be a sterile or sterilizable liquid, such as a water (e.g., water for injection) or a natural or synthetic oil (e.g., a petroleum-based or mineral oil, an animal oil, or a vegetable oil (e.g., a peanut, soybean, sesame, or canola oil)). The carrier can also be a solid; a liquid that includes one or more solid components (e.g., a salt, for example, a “normal saline”); a mixture of solids; or a mixture of liquids.

The term “comparable” refers to two or more items (e.g., agents, entities, situations, sets of conditions, etc.) that are not identical to one another but are sufficiently similar to permit comparison therebetween so that one of ordinary skill in the art will appreciate that conclusions may reasonably be drawn based on differences or similarities observed. In some embodiments, comparable sets of conditions, circumstances, individuals (e.g., an individual patient or subject), or populations are characterized by a plurality of substantially identical features and one or a small number of varied features. One of ordinary skill in the art will understand, in context, what degree of identity is required in any given circumstance for two or more items to be considered comparable. For example, two items are comparable to one another when they have in common a sufficient number and type of substantially identical features to warrant a reasonable conclusion that any differences in results obtained or phenomena observed with the items are caused by or are indicative of the variation in those features that are varied. In some embodiments, a comparable item serves as a “control.” For example, a “control subject/population” can be an untreated (or placebo-treated) individual/population who/that is afflicted with the same disease as an individual/population being treated.

The term “combination therapy” refers to those situations in which a subject is exposed to two or more therapeutic regimens (e.g., two or more therapeutic agents (e.g., three agents)) to treat a single disease (e.g., a cancer as described herein). The two or more regimens/agents may be administered simultaneously or sequentially. When administered simultaneously, a dose of the first agent and a dose of the second agent are administered at about the same time, such that both agents exert an effect on the patient at the same time or, if the first agent is faster- or slower-acting than the second agent, during an overlapping period of time. When administered sequentially, the doses of the first and second agents are separated in time, such that they may or may not exert an effect on the patient at the same time. For example, the first and second agents may be given within the same hour or same day, in which case the first agent would likely still be active when the second is administered. Alternatively, a much longer period of time may elapse between administration of the first and second agents, such that the first agent is no longer active when the second is administered (e.g., all doses of a first regimen are administered prior to administration of any dose(s) of a second regimen by the same or a different route of administration, as may occur in treating a refractory cancer). For clarity, combination therapy does not require that individual agents be administered together in a single composition or at the same time, although in some embodiments, two or more agents, including a compound of the present disclosure and a second agent described herein, may be administered within the same period of time (e.g., within the same hour, day, week, or month).

The terms “dosage form,” “formulation,” and “preparation” refer to compositions that contain a compound described herein (e.g., tamibarotene, an HMA, or a BCL2 inhibitor), or to other biologically or therapeutically active ingredients suitable for use as described herein (e.g., in combination with tamibarotene). The term “unit dosage form” refers to a physically discrete unit of or containing a compound described herein (e.g., tamibarotene) or a pharmaceutically acceptable salt thereof. One or more of an additional/second agent can also be formulated, administered, or used as described herein in a unit dosage form. Each such unit can contain a predetermined quantity of the active pharmaceutical ingredient, which may be the amount prescribed for a single dose (i.e., an amount expected to correlate with a desired outcome when administered as part of a therapeutic or prophylactic regimen) or a fraction thereof (e.g., a unit dosage form (e.g., a tablet or capsule) may contain one half of the amount prescribed for a single dose, in which case a patient would take two unit dosage forms (i.e., two tablets or two capsules)). One of ordinary skill in the art will appreciate that the total amount of a composition or agent administered to a particular subject is determined by one or more attending physicians and may involve administration of multiple unit dosage forms (e.g., as described herein).

The term “dosing regimen” refers to the unit dosage form(s) administered to, or prescribed for, a patient, and typically includes more than one dose separated by periods of time (e.g., as described herein or known in the art). The dosage form(s) administered within a dosing regimen can be of the same unit dose amount or of different amounts. For example, a dosing regimen can include a first dose in a first dose amount, followed by one or more additional doses in a second dose amount that is the same as or different from the first dose amount.

An “effective amount” refers to an amount of an agent (e.g., tamibarotene or a “second” agent as described herein) that produces the desired effect for which it is administered. In some embodiments, the term refers to an amount that is sufficient, when administered to a population suffering from or susceptible to a disease in accordance with a therapeutic dosing regimen, to treat the disease, in which case the effective amount may also be referred to as a “therapeutically effective amount.” One of ordinary skill in the art will appreciate that a therapeutically effective amount may not achieve a successful treatment in any particular individual (i.e., in any given individual patient). Rather, a therapeutically effective amount provides a desired pharmacological response in a significant or certain expected number of subjects when administered to a population of patients in need of such treatment. A reference to an effective amount may be a reference to an amount of an agent administered or an amount measured in one or more specific tissues (e.g., a tissue affected by the disease) or fluids (e.g., blood, saliva, urine, etc.) after administration.

“Improve(s),” “increase(s)” or “reduce(s)/decrease(s)” (and obvious variants thereof, such as “improved” or “improving”) are terms used to characterize the manner in which a value changes or has changed relative to a reference value. For example, a measurement obtained from a patient (or a biological sample obtained therefrom) prior to treatment can be increased or reduced/decreased relative to that measurement when obtained during or after treatment in the same patient, a control patient, on average in a patient population, or in biological sample(s) obtained therefrom. The value may be improved in either event, depending on whether an increase or decrease is associated with a positive therapeutic outcome.

A “patient” to which administration is contemplated as described herein includes, but is not limited to, humans, and the patient may be a male, female, transgendered or other-gendered person (e.g., a child, adolescent, or adult subject (e.g., young adult, middle-aged adult, or senior adult (e.g., an elderly patient aged 65 or older)). Elderly patients may be deemed unfit for standard induction chemotherapy and so may be referred to as an “elderly unfit” patient.

A “pharmaceutical composition” or “pharmaceutically acceptable composition,” which we may also refer to as a “pharmaceutical formulation” or “pharmaceutically acceptable formulation,” is a composition/formulation in which an active agent (e.g., an active pharmaceutical ingredient (e.g., tamibarotene or second agent as described herein)) is formulated together with one or more pharmaceutically acceptable carriers. The active agent/ingredient can be present in a unit dose amount appropriate for administration in a therapeutic regimen that shows a statistically significant probability of achieving a predetermined therapeutic effect when administered to a relevant population. The pharmaceutical composition may be specially formulated for administration in solid or liquid form, including such forms made for oral or parenteral administration. For oral administration, the pharmaceutical composition can be formulated, for example, as an aqueous or non-aqueous solution or suspension or as a tablet or capsule. For systemic absorption through the mouth, the composition can be formulated for buccal administration, sublingual administration, or as a paste for application to the tongue. For parenteral administration, the composition can be formulated, for example, as a sterile solution or suspension for subcutaneous, intramuscular, intravenous, intra-arterial, intraperitoneal, intra-tumoral, or epidural injection. Pharmaceutical compositions comprising an active agent/ingredient (e.g., a compound described herein or a specified form thereof) can also be formulated as sustained-release formulations or as a cream, ointment, controlled-release patch, or spray for topical application. Creams, ointments, foams, gels, and pastes can also be applied to mucus membranes lining the nose, mouth, vagina, and rectum. Any of the compounds described herein and any pharmaceutical composition containing such a compound may also be referred to as a “medicament.”

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 66:1-19, 1977. Pharmaceutically acceptable salts of the compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, MALAT1e, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “population of patients” designated for treatment according to the methods described herein is two or more patients (e.g., 2, 5, 10, about 100, or about 1000) patients.

The term “RARA gene” refers to a genomic DNA sequence that encodes a functional retinoic acid receptor-α (RARA) and specifically excludes gene fusions that comprise all or a portion of the RARA gene. In some embodiments, the RARA gene is located at chr17:38458152-38516681 in genome build hg19.

The term “reference” describes a standard or control relative to which a comparison is made. For example, an agent, animal (e.g., a subject (e.g., an animal used in laboratory studies)), cell or cells, individual (e.g., an individual patient), population, sample (e.g., biological sample), sequence or value of interest is compared with a reference or control agent, animal (e.g., a subject (e.g., an animal used in laboratory studies)), cell or cells, individual (e.g., an individual patient), population, sample, or sequence or value, respectively. In some embodiments, a reference or control is tested and/or determined substantially simultaneously with the testing or determination of interest. In other embodiments, a reference or control is a historical reference or control, optionally embodied in a tangible medium. Typically, as would be understood by one of ordinary skill in the art, a reference or control is determined or characterized under comparable conditions to those under assessment, and one of ordinary skill in the art will appreciate when sufficient similarities are present to justify reliance on and/or comparison to a particular possible reference or control.

The term “response” with respect to a treatment may refer to any beneficial alteration in a patient's condition that occurs as a result of, or correlates with, treatment. Such an alteration may include stabilization of the condition (e.g., prevention of deterioration that would have taken place in the absence of the treatment (e.g., stable disease)), amelioration of symptoms of the condition, and/or improvement in the prospects for cure of the condition (e.g., tumor regression), etc. The response may be a cellular response (e.g., as assessed in a cancer cell) and can be measured using a wide variety of criteria, including clinical criteria and objective criteria, known in the art. Techniques for assessing a response include, but are not limited to, assay assessment, clinical examination, positron emission tomography, X-ray, CT scan, MM, ultrasound, endoscopy, laparoscopy, assessing the presence or level of tumor markers in a sample obtained from a subject, cytology, and/or histology. Regarding measuring tumor response, methods and guidelines for assessing response to treatment are discussed in Therasse et al. (J. Natl. Cancer Inst., 92(3):205-216, 2000). The exact response criteria can be selected by one of ordinary skill in the art in any appropriate manner, provided that when comparing groups of cancers and/or patients, the groups to be compared are assessed based on the same or comparable criteria for determining response rate.

Other terms and abbreviations that may be used herein include AE for adverse event; AML for acute myeloid leukemia; APL for acute prolyelocytic leukemia; ATRA for all-trans retinoic acid; BID for twice per day; BSA for body surface area; CI for confidence interval; CR for complete remission or complete response; DOCR for duration of complete response; EFS for event-free survival; HR-MDS for higher-risk myelodysplastic syndrome; ORR for overall response rate; OS for overall survival; PD for progressive disease; PK for pharmacokinetic; PR for partial remission or partial response; RARA or RARα for retinoic acid receptor alpha; RBC for red blood cell; R/R for relapsed/refractor; SAE for serious adverse event; TI for transfusion independence; and ULN for upper limit of normal.

Patients amenable to treatment with tamibarotene or a combination of tamibarotene and one or more additional therapeutic agents, as described herein: In one embodiment, the present methods comprise administering tamibarotene or a therapeutically effective, pharmaceutically acceptable salt thereof, alone or in combination with one or more of the additional (e.g., second, third, or fourth) therapeutic agents described herein. For example, tamibarotene at the fixed dose of about 8-14 mg/day (e.g., 12 mg/day), can be administered in combination with an HMA, such as azacitidine or decitabine, optionally with the further inclusion of venetoclax, optionally with the further inclusion of low-dose ara-C(LDAC) (e.g., for patients with an AML such as non-APL AML), obinutuzumab (e.g., for patients with CLL or SLL), rituximab (e.g., for patients with CLL or SLL, with or without 17p deletion), a CD47 inhibitor (e.g., magrolimab) (e.g., for patients with an MDS), or an endocrine therapy (e.g., an androgen therapy for patients with persistent anemia). For example, tamibarotene, tamibarotene in combination with an HMA (e.g., azacitidine or decitabine), or tamibarotene in combination with an HMA (e.g., azacitidine or decitabine) and venetoclax, optionally further including LDAC, can be administered to newly diagnosed AML patients (e.g., of the M4 or M5 subtype (identified by any method known in the art, including by virtue of a monocyte/monocytic expression signature (MES) as described herein). The patient can be at least 60 years old (e.g., at least 60, 65, 70, or 75 years or older) and/or can have comorbidities that preclude use of intensive induction chemotherapy (i.e., the patient can be deemed unfit). In another embodiment, the methods comprise administering tamibarotene alone or in combination with one or more “second” therapeutic agents as just described to a patient who has relapsed following treatment with venetoclax, has become refractory to treatment with venetoclax, or whose cancer cells demonstrate resistance to venetoclax (e.g., by an ex vivo assay; e.g., a patient having CLL or SLL). Thus, in one embodiment, the methods of the invention comprise administering tamibarotene, at 8-14 mg/day (e.g., 12 mg/day), alone or in combination with venetoclax, optionally also including an HMA (e.g., azacitidine or decitabine) and/or obinutuzumab to a patient (e.g., an ND patient) who has CLL or SLL. In another embodiment, the methods comprise administering tamibarotene, at 8-14 mg/day (e.g., 12 mg/day), alone or in combination with venetoclax, optionally also including an HMA (e.g., azacitidine or decitabine) and/or rituximab to a patient (e.g., an ND patient) who has CLL or SLL. In any one or more of these embodiments, the tamibarotene or a therapeutically effective, pharmaceutically acceptable salt thereof, can be administered orally, in a single or divided daily dose, and/or on specified days of a treatment cycle (e.g., on days 8-28 of a 28-day treatment cycle). In general, each therapeutic agent for use as described herein (e.g., as a second agent, third agent, or fourth agent) is formulated, dosed, and administered in a therapeutically effective amount using pharmaceutical compositions and dosing regimens that are consistent with good medical practice and appropriate for the relevant agent(s) and patient(s). Where tamibarotene is administered in combination with one or more second therapeutic agents, those agents can be administered according to a dosing regimen for which they are approved for individual use. In the treatment regimen as a whole, the HMA can be administered to a patient having a hematopoietic cancer (e.g., to a patient having AML (e.g., a relapsed or refractory AML) or an MDS (e.g., a higher risk MDS)), e.g., in a dose and by a route described herein or known in the art, on days 1-7 of the treatment regimen (typically by an intravenous or subcutaneous route), and tamibarotene can be administered, e.g., orally at a dose of about 8-14 mg/day (e.g., 12 mg/day), on days 8-28 of the treatment regimen, after which treatment could cease, be repeated, or after which the patient could have a reprieve from treatment for a period of days or weeks.

With regard to higher risk MDS, and as referenced elsewhere herein, the IPSS-R uses five prognostic indicators to predict the course of patient disease: (1) blasts, (2) cytogenetics, (3) hemoglobin, (4) platelet count, and (5) absolute neutrophil count. A score of 1.5 or fewer points indicates very low risk; a score greater than 1.5 and up to and including 3 points indicates low risk; a score of greater than 3 and up to and including 4.5 points indicates intermediate risk; a score of greater than 4.5 and up to and including 6 points indicates high risk; and a score of 6.5 or more points indicates very high risk. Based on the IPSS-R, a patient has higher risk MDS when they have a score of more than 4.5 points. WPPS is not used as often as IPSS-R. It differs from IPSS-R in that it includes the MDS subtype as a prognostic factor. It also assigns a score based on the presence or absence of severe anemias. In the WPPS, a score of 0 points indicates very low risk, a score of 1 point indicates low risk, a score of 2 points indicates intermediate risk, a score of 3 to 4 points indicates high risk, and a score of 5 to 6 points indicates very high risk. Based on the WPSS, a patient has higher risk MDS when they have a score of more than 3 points (see Lorand-Metze et al., Hematol. Transfus. Cell Ther., 40(3):262-266, 2018).

Tamibarotene: Tamibarotene is an orally available, synthetic retinoid approved in Japan since April 2005 for the treatment of relapsed or refractory APL, characterized by the presence of the t(15;17) translocation or expression of the PML-RARα gene (see, e.g., Miwako, supra, and Tobita et al., supra). Tamibarotene was designed to be a more potent and selective RARα agonist with significantly improved in vivo pharmacologic properties compared to ATRA, a component of the current first-line treatment of APL. In vitro, tamibarotene is approximately 10-fold more potent than ATRA. Tamibarotene has the structure:

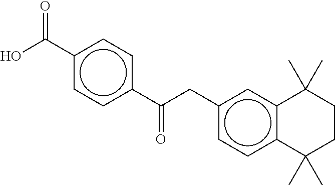

and is also known as AM-80, INNO-507, NSC-608000, OMS-0728, TM-411, TOS-80, TOS-80T, and Z-208.

Azacitidine and Decitabine: Azacitidine is a pyrimidine analogue that exerts antineoplastic effects on abnormal hematopoietic bone marrow cells through multiple mechanisms including deoxyribonucleic acid (DNA) hypomethylation. Cytotoxicity may also result from incorporation into DNA and ribonucleic acid (RNA), with inhibition of DNA, RNA, and protein synthesis. Azacitidine is approved in the United States (US) and European Union (EU) for treatment of MDS. Azacitidine is also approved for use in AML in the EU and is widely accepted as the standard of care for treatment of AML in the US. Decitabine is an analog of cytidine and is also a hypomethylating agent. It is used for the treatment of myelodysplastic syndromes and AML.

Other HMAs: In addition to azacitidine and decitabine, other hypomethylating agents (HMAs) that can be used as the “second” therapeutic agent in the methods described herein include guadecitabine (also known as SGI-110, a cytosine analog), FdCyd (also known as 5-fluro-2′-deoxycytidine, another cytosine analog), zebularine (a cytosine analog), CP-4200 (a cytosine analog), RG108 (a small molecule inhibitor), nanaomycin A (a small molecule inhibitor), and lenalidomide.

Venetoclax and other BCL2 Inhibitors: BCL2 inhibitors, including those approved for use or in clinical-stage development, can be employed in the methods and uses described herein. More specifically, venetoclax is available and can be administered in tablet form, each dosage unit containing either 10, 50, or 100 mg of the therapeutic agent. Where venetoclax is administered in combination with tamibarotene and an HMA (e.g., azacitidine) to treat a hematological cancer (e.g., CLL or SLL), the venetoclax can be dosed according to a weekly ramp-up schedule over a period of weeks (e.g., five weeks) to the recommended daily dose of 400 mg. For example, a patient with a hematological cancer (e.g., CLL or SLL) may receive a combination therapy as described herein that includes venetoclax at 20 mg, PO, QD in week 1; venetoclax at 50 mg, PO, QD in week 2; venetoclax at 100 mg PO, QD in week 3; venetoclax at 200 mg PO, QD in week 4; and venetoclax at 400 mg PO, QD in week five and beyond. Modified versions of this treatment regimen are known in the art for subsequent cycles of treatment (e.g., cycle 2, cycles 3-6, and cycles 7-12). Dosing of venetoclax can be as described in any one or more of U.S. Pat. Nos. 8,546,399; 8,722,657; 9,174,982; 9,539,251; 10,730,873; and 10,993,942, which are hereby incorporated by reference herein in their entireties. Alternatively, the second-generation BCL2 inhibitor S65487 can be administered (e.g., intravenously administered) and may be especially well suited for administration to patients diagnosed with AML, NHL, MM, and CLL. Alternatively, the selective BCL2 inhibitor BGB-11417 can be administered (e.g., orally administered once daily) and may be especially well suited for administration to patients diagnosed with relapsed/refractory NHL, CLL, or SLL.

Alternatively, the BCL-XL/BCL2 inhibitor navitoclax (ABT-263) can be administered (e.g., by way of an oral tablet or solution dose of 150 mg lead-in dose for 7-14 days followed by a 325 mg continuous once daily dose) and may be especially well suited for patients diagnosed with NHL or CLL. Alternatively, the BCL2 inhibitor pelcitoclax (APG-1252) can be administered (e.g., twice per week (BIW) or once per week (QW) at a dose ranging from 10 to 400 mg in a 28-day cycle) and may be especially well suited for patients diagnosed with NHL. Alternatively, the BCL2 inhibitor lisaftoclax (APG-2575) can be administered (e.g., orally at doses ranging from 20 to 1,200 mg) and may be especially well suited for patients diagnosed with R/R CLL.

For administration (either oral or parenteral administration), a therapeutic agent described herein can be readily formulated by combining the agent with one or more pharmaceutically acceptable carriers, which are well known in the art. Indeed, formulations of RARA agonists (e.g., tamibarotene), HMAs (e.g., azacitidine and decitabine), and BCL2 inhibitors (e.g., venetoclax) are known in the art and can be employed in the methods (or used) as described herein. For example and as noted above, venetoclax is available in tablet form, each dosage unit containing either 10, 50, or 100 mg of the therapeutic agent, and such tablets can be employed in any of the methods (or used as) described herein. For example, where venetoclax is administered in combination with tamibarotene and an HMA (e.g., azacitidine) to treat a hematological cancer (e.g., CLL or SLL), the venetoclax can be dosed according to a weekly ramp-up schedule over a period of weeks (e.g., five weeks) to the recommended daily dose of 400 mg.

LDAC: Low-dose ara-C(available under the brand name cytarabine) can be employed together with tamibarotene as described herein and may be particularly useful when the hematopoietic cancer is AML (e.g., APL, the M4 or M5 subtype of AML, or non-APL AML), CML, or ALL. LDAC is administered by intravenous or intrathecal infusion, and it may be administered as such according to known dosing schedules in the present methods.

Obinutuzumab: Where a method (or use) described herein includes administration of obinutuzumab, in addition to venetoclax, tamibarotene, and an HMA (e.g., azacitidine), the obinutuzumab can be administered intravenously at 100 mg on day 1; at 900 mg on day 2; and at 1,000 mg on days 8 and 15 prior to a ramp up dosing schedule with venetoclax. Modified versions of this treatment regimen are known in the art for subsequent cycles of treatment (e.g., cycle 2, cycles 3-6, and cycles 7-12).

Hematopoietic cancers: The methods described herein can be applied to patients having a hematopoietic cancer (e.g., a leukemia, a lymphoma, multiple myeloma, or myeloid neoplasm with myelodysplasia (e.g., a myelodysplastic syndrome (MDS)). HR-MDS is an orphan disease with a high unmet need for effective, tolerable therapies. Allogeneic HSCT (hematopoietic stem cell transplantation) is the only curative treatment for this condition. While azacitidine is the standard of care for newly diagnosed HR-MDS patients, CR and PR rates are low, and treatment is not curative. Thus, novel therapies that replace or augment the efficacy of azacitidine are needed to extend survival of patients with HR-MDS. Furthermore, no standard of care exists for patients who fail frontline therapy with hypomethylating agents. Thus, patients with HR-MDS have a poor prognosis and a need for more effective treatment options.

Where the hematopoietic cancer is AML, it may be a subtype thereof, such as the M4 or M5 subtype, also known as acute myelomonocytic leukemia and acute monocytic leukemia, respectively. As is understood in the art, cells affected by AML do not usually form tumors but, instead, are generally found throughout the bone marrow and can be found in other organs (e.g., the spleen and liver). Two organizations, the French-American-British (FAB) collaboration and the World Health Organization (WHO), have developed classifications for various subtypes of AML. The FAB defined eight subtypes (M0 through M7) based on the cell type in which the cancer originated and the maturity of the cells, based in large part on their appearance under the microscope after staining. The FAB subtypes are: M0 (undifferentiated acute myeloblastic leukemia); M1 (acute myeloblastic leukemia with minimal maturation); M2 (acute myeloblastic leukemia with maturation); M3 (acute promyelocytic leukemia (APL)); M4 (acute myelomonocytic leukemia); M4 eos (acute myelomonocytic leukemia with eosinophilia); M5 (acute monocytic leukemia); M6 (acute erythroid leukemia); and M7 (acute megakaryoblastic leukemia). The M0 through M5 subtypes originate in immature forms of white blood cells; the M6 subtype originates in immature forms of red blood cells; and the M7 subtype originates in immature platelets. The WHO system includes factors known to affect prognosis, and references AML with certain genetic abnormalities and AML with myelodysplasia-related changes. The genetic abnormalities include APL with the PML-RARA fusion gene and AML with: a translocation between chromosomes 8 and 21 [t(8;21)]; a translocation or inversion in chromosome 16 [t(16;16) or inv(16)]; a translocation between chromosomes 9 and 11 [t(9;11)]; a translocation between chromosomes 6 and 9 [t(6;9)]; a translocation or inversion in chromosome 3 [t(3;3) or inv(3)]; a translocation between chromosomes 1 and 22 [t(1:22)]; the BCR-ABL1 (BCR-ABL) fusion gene (currently considered a provisional entity, as there may not be enough evidence this is a unique subtype); a mutated NPM1 gene; biallelic mutations of the CEBPA gene; mutations of the RUNX1 gene (also a provisional entity). Hematopoietic cancers of any of these subtypes are amenable to treatment with the fixed dose of tamibarotene described herein.

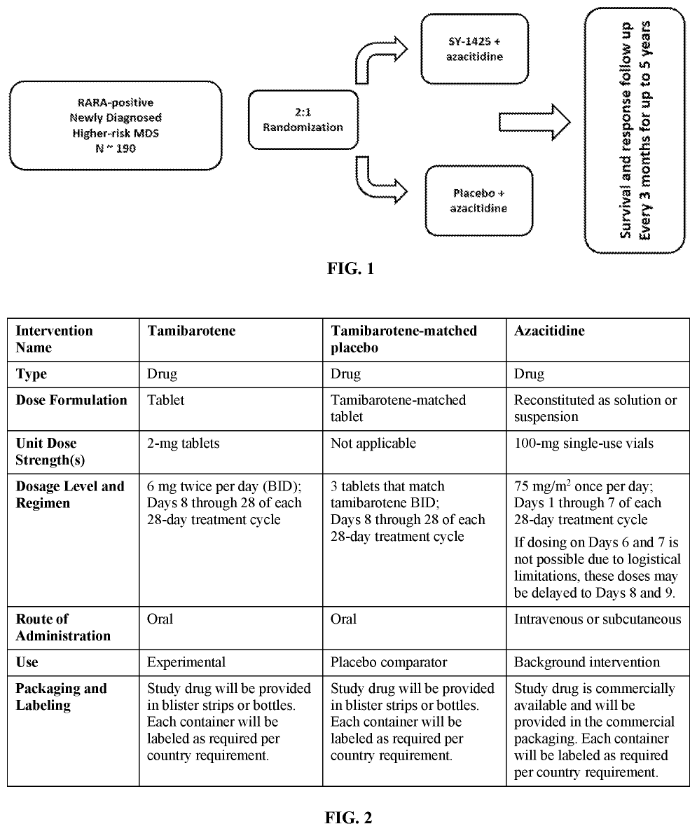

MDS and AML are related hematological disorders associated with impaired differentiation of myeloid progenitor cells. Myelodysplastic syndromes are known to arise from the growth and spread of a somatically mutated clone of hematopoietic cells, which frequently evolves into AML (see, for example, FIG. 1 of Cazzola, New Engl. J. Med. 383:1358-1374, 2020, and the description of leukemic transformation at page 1363).

Other specific hematopoietic cancers amenable to treatment as described herein (i.e., with a fixed dose of tamibarotene (of, for example, about 8-14 mg/day (e.g., 12 mg per day), orally, optionally in divided daily doses (e.g., BID))) include: myelodysplastic-myeloproliferative neoplasms such as chronic myelomonocytic leukemia (CMML); chronic lymphocytic leukemia (CLL (e.g., with 17p deletion)); acute lymphoblastic leukemia (ALL); small lymphocytic lymphoma (SLL); multiple myeloma (MM); Hodgkin lymphoma (HL); non-Hodgkin lymphoma (NHL); and mantle cell lymphoma (MCL). Other subtypes of myelodysplastic-myeloproliferative neoplasms amenable to treatment include BCR-ABL1-negative atypical chronic myeloid leukemia, myelodysplastic-myeloproliferative neoplasm with ring sideroblasts and thrombocytosis, and juvenile myelomonocytic leukemia.

MES: As noted, AML patients of the M4 or M5 subtype can be identified by any method known in the art, including by virtue of a monocyte/monocytic expression signature (MES). In determining that signature, one can assay a biological sample obtained from the patient for elevated expression, relative to a reference, of a primary RNA transcript from one or more of the genes CD14, CLEC7A (CD369), CD86, CD68, LYZ, MAFB, CD34, ITGAM (CD11b), FCGR1A (CD64), RARA, and KIT (CD117) (e.g., the expression levels of KIT, CD64, CD86, and LYZ)), or a protein encoded thereby, and/or a correlate thereof (e.g., elevated expression of MCL1 and/or under expression of BCL2). For example, the biomarker of the M4/M5 subtype can be the expression level of a combination of the genes CD34, KIT (CD117), and BCL2. One can also assess the enhancer or super enhancer associated with a gene that contributes to the IVIES by techniques known in the art.

In clinical studies, the Applicant has explored, and is continuing to explore, the safety, pharmacokinetic, pharmacodynamic, and clinical activity of tamibarotene in non-APL AML in combination with azacitidine. Clinical activity data support the rationale for further development of the combination of tamibarotene with azacitidine in a genomically defined subset of patients characterized by overexpression of RARA in blasts. HR-MDS is biologically closely related to AML, with common mutations found across these conditions (Arber et al., Blood 127(20):2391-2405, 2016), supporting the hypothesis that the combination of tamibarotene plus azacitidine will have clinical activity in those with newly diagnosed HR-MDS.

The AE profile of the combination was consistent with what has been previously reported for single agent tamibarotene or single agent azacitidine in treatment of AML/MDS patients. The rates of myelosuppression with tamibarotene plus azacitidine combination were comparable to single agent azacitidine, without evidence for additive hematologic toxicity from the combination. The majority of nonhematologic AEs were low grade and reversible. The safety profile of tamibarotene plus azacitidine is further supported by the Japanese marketing experience with tamibarotene, in which more than 1000 patients have been treated for R/R APL. Overall, the safety profile of tamibarotene is considered acceptable, with the benefit to the patient primarily defined by the potential clinical utility of receiving tamibarotene plus azacitidine for the duration of the study (for patients randomized to the active combination). Both treatment arms of the randomized study contain azacitidine, which is the current standard of care for treating HR-MDS.

The risks of the underlying conditions evaluated in this study are significant and represent areas of high unmet medical need. HR-MDS is associated with the risk of infection and complications due to persistent cytopenias. Patients undergoing treatment for HR-MDS undergo the procedural risk of repeated bone marrow aspirations, which are also required assessments in this study. The clinical activity of tamibarotene plus azacitidine, coupled with the emerging favorable safety profile of tamibarotene plus azacitidine combination, suggests that this combination has the potential to provide a valuable treatment option for RARA-positive patients with AML, as well as those with HR-MDS.

### EXAMPLES

### Example 1: A Randomized, Double-Blind, Placebo-Controlled Phase 3 Study of Tamibarotene Plus Azacitidine Versus Placebo Plus Azacitidine in Newly Diagnosed, RARA-Positive Adult Patients with Higher-Risk Myelodysplastic Syndrome (a Prophetic Example)

Patients are eligible to be included in the study only if all of the pre-determined criteria apply. These include age (at least 18 years old at the time of signing of an informed consent); RARA biomarker status (positive); a diagnosis of HR-MDS; measurable disease with bone marrow blasts>5% at the screening visit; and adequate organ function. Diagnosis with HR-MDS requires meeting the World Health Organization (WHO) classification and that by the Revised International Prognostic Scoring System (IPSS-R) risk category as: (a) very high (risk score>6), high (risk score>4.5 to 6), or intermediate (risk score>3 to 4.5). Adequate organ function, as defined by: (a) total bilirubin≤3.0× the upper limit of normal (ULN), (b) alanine aminotransferase (ALT) and aspartate aminotransferase (AST)≤3×ULN, and (c) creatinine clearance≥30 mL/min based on the Cockcroft-Gault Glomerular Filtration Rate estimation. Patients are excluded from the clinical trial where any pre-determined criteria is met (e.g. if a patient has received prior treatment for MDS with an HMA, previously received ATRA, is immunocompromised, or pregnant). The proposed eligibility criteria are intended to ensure that only newly diagnosed, RARA-positive patients with HR-MDS are enrolled in the study. Allogeneic HSCT is the curative therapy for HR-MDS; therefore, patients who are transplant-eligible at the time of screening and agree to undergo this treatment will be excluded. Patients who received prior treatment with hypomethylating agents or chemotherapy (including lenalidomide) are excluded to avoid confounding of interpretation of the effect of study treatment. Transformation of MDS into secondary AML is defined by either an increase in the bone marrow blast count of ≥50% and a total marrow blast count of ≥20% or a ≥20% blast count in the peripheral blood.

This is a Phase 3, multi-center, randomized, double-blind, placebo-controlled study comparing the activity of tamibarotene plus azacitidine to placebo plus azacitidine as first line of therapy in RARA-positive patients with newly diagnosed HR-MDS.

At the Pre-screening Visit, blood samples will be collected and sent to a laboratory, currently expected to be the Almac Diagnostics Laboratory, for assessment of RARA biomarker to determine study eligibility. Pre-screening and screening assessments will be performed within 30 days of Cycle 1, Day 1.

Patients will be randomized 2:1 to receive either tamibarotene plus azacitidine or placebo plus azacitidine. Randomization will be stratified by the IPSS-R risk group (Intermediate, High, and Very High Risk).

Response will be assessed by the investigator per the modified IWG MDS criteria (Cheson, 2006). Bone marrow aspirates will be collected to measure response on Day 1 of Cycles 2 and 4, followed by every third cycle (7, 10, 13, etc.), with bone marrow aspirates collected at other times as clinically determined based upon clinical findings or changes in peripheral blood counts. Following the response assessment on Day 1 of Cycle 7, those in CR may have the frequency of bone marrow aspirates for response assessment reduced to every 6 cycles.

Patients will undergo response and safety evaluations throughout their study participation as detailed in the SoA. Patients may continue to receive study drug until experiencing an unacceptable toxicity, disease progression (including transformation into AML), relapse, decision to pursue post-remission therapy (such as HSCT) or an alternative anticancer therapy, patient withdraws consent, or the investigator determines it is in the best interest of the patient to discontinue study drug.

An EoT Visit should occur within 3 days of the last dose of study drug (or within 3 days of decision to permanently stop drug treatment, if this decision directly follows a period when study drug has been on hold) and before the start of any subsequent anticancer therapy. A Safety Follow-up Visit should occur 30 days (±3 days) after the EoT Visit and before the start of any subsequent anticancer therapy. After the EoT Visit, patients who have not progressed/relapsed or initiated subsequent anticancer therapy (not study drug) will be followed every 3 months for response assessment for up to 5 years, until the initiation of subsequent anticancer therapy, or until disease progression/relapse, whichever occurs first. All patients will be followed to document the start of subsequent anticancer therapy, transformation to AML, and OS every 3 months for up to 5 years after discontinuation of study drug. A schematic of the study design is shown in FIG. 1 and a table highlighting details of the therapeutic agents is shown in FIG. 2.

A total of 6 tablets (12 mg total) will be taken each day, 3 tablets (6 mg) in the morning and 3 tablets (6 mg) in the evening. The selection of a 6 mg BID regimen of tamibarotene administered on days 8 to 28 of a 28-day treatment cycle is based on the totality of safety, efficacy, and PK data available to us for tamibarotene to date, including from a Phase 2 study of tamibarotene in adults with AML or MDS and the Japanese clinical trial and marketing experience.

The dose and regimen used in a prior study (6 mg/m²/day in 2 divided doses) were based on the dose and regimen approved in Japan for the treatment of APL. Population PK analysis from that study indicated that body size was not significantly associated with clearance; therefore, a body surface area (BSA)-based adjustment to tamibarotene dosing is not necessary. Based on this analysis, the fixed dosing regimens described herein, including the 6 mg BID flat dosing regimen, are expected to provide similar PK exposures compared to the 6 mg/m²/day (in 2 divided doses) regimen used in the prior study.

### Example 2: The Rationale for “Flat” Also Known as “Fixed” Dose Administration of Tamibarotene (i.e., Dosing Independent of Patient Weight or BSA)

We have found no evidence that the efficacy of tamibarotene, observed in prior studies in which that agent was administered at 6 mg/m²/day, would be compromised by flat- or fixed-amount dosing. This was unexpected and is beneficial (because, for example, it may lead to better patient compliance and fewer dosing errors by both physicians and patients).

We have analyzed about 900 samples from patients previously treated with tamibarotene on the basis of BSA. A population PK model was developed using patient characteristics, and we assessed the impact of weight on drug clearance. Simulations were conducted to assess the performance of “flat” versus BSA-based dosing. Simulated AUC (area-under-the-curve) from the model was used to establish relationship of exposure with efficacy and safety; we analyzed exposure and achievement of CR/CRi and exposure with adverse events equal to or greater than those of grade 3. In our models, we found that flat dosing of 12 mg of tamibarotene per day provided similar exposures as BSA-based dosing for daily doses of 10-14 mg; the models predict that flat dosing of 12 mg administered to patients regardless of BSA will achieve exposures similar to those obtained using the 6 mg/m 2 labeled dosing regimen. The population PK modeling and simulations demonstrated that similar exposure to tamibarotene was achieved with either BSA-based or flat daily dosing across the dose range of 10 to 14 mg/day doses, and that 12 mg flat daily dosing (administered as 6 mg BID) resulted in similar exposures across the BSA ranges. Higher AUCs were not associated with a higher probability of achieving CR.

### Example 3: Tamibarotene in Combination with Venetoclax and Azacitidine in Previously Untreated Adult Patients Selected for RARA-Positive AML Who are Ineligible for Standard Induction Therapy (a Prophetic Example)

Currently, ND AML, patients who are ineligible for standard induction therapy are increasingly receiving the combination of venetoclax and azacitidine as an initial treatment. In a randomized study of such AML patients, the combination of azacitidine with venetoclax has demonstrated a high composite complete remission (CR) rate and a survival benefit compared to treatment with azacitidine monotherapy (DiNardo et al., N. Engl. J. Med. 383(7):617-629, 2020). However, approximately one-third of patients failed to respond to the venetoclax/azacitidine combination (DiNardo et al., supra), supporting the existing need to improve upon the outcomes in this subpopulation.

In a phase 2, open-label, 3-part, multi-center study, we will evaluate tamibarotene-venetoclax-azacitidine combination therapy in RARA-positive, previously untreated, non-APL AML, patients who are unlikely to tolerate standard intensive chemotherapy at the time of study entry. Azacitidine will be administered intravenously or subcutaneously at 75 mg/m 2 each day, on days 1 through 7 of each 28-day therapy cycle (per VIDAZA United States Prescribing Information (USPI)). Alternative dosing of azacitidine (days 1-5, 8, and 9) will be permitted throughout the study. Venetoclax will be administered orally at a dose described in the most current VENCLEXTA USPI and VENCLYXTO SmPC, including ramp-up and appropriate dosing for patients receiving concomitant CYP3A and P-gp inhibitors, daily on days 1 through 28. Tamibarotene will be administered orally at 6 mg twice per day (BID) on days 8 through 28 or each 28-day therapy cycle.

What is claimed is:

1. A method of treating a patient who has a hematopoietic cancer, the method comprising administering to the patient about 12 mg of tamibarotene per day.

2. The method of claim 1, wherein the about 12 mg of tamibarotene is divided into two doses, the first being a first daily dose and the second being a second daily dose.

3. The method of claim 2, wherein the first daily dose and the second daily dose each contain about 6 mg of tamibarotene.

4. The method of claim 1, wherein the tamibarotene is administered orally.

5. The method of any one of claims 1-4, wherein the about 12 mg of tamibarotene is administered regardless of the patient's weight or body surface area and/or wherein the patient is a child, adolescent, or adult human patient.

6. The method of any one of claims 1-4, wherein the patient is an adult human.

7. The method of any one of claims 1-4, wherein the hematopoietic cancer is a leukemia, optionally a leukemia resistant or refractory to treatment with venetoclax, a lymphoma, multiple myeloma, or myeloid neoplasm with myelodysplasia.

8. The method of claim 7, wherein the leukemia is acute lymphoblastic leukemia (ALL), chronic lymphocytic leukemia (CLL), acute myeloid leukemia (AML), or chronic myeloid leukemia (CML), and the lymphoma is Hodgkin lymphoma or non-Hodgkin lymphoma.

9. The method of claim 8, wherein the AML is non-acute promyelocytic leukemia acute myeloid leukemia (non-APL AML).

10. The method of claim 7, wherein the myeloid neoplasm with myelodysplasia is a myelodysplastic syndrome (MDS).

11. The method of claim 10, wherein the MDS is higher risk MDS.

12. The method of any one of claims 1-4, wherein the method comprises administering a second therapeutic agent, wherein the second therapeutic agent is a hypomethylating agent (HMA) or a BCL2 inhibitor.

13. The method of claim 12, wherein the HMA is decitabine or azacitidine and the BCL2 inhibitor is venetoclax.

14. The method of claim 13, wherein the HMA is azacitidine.

15. The method of claim 14, wherein the azacitidine is administered parenterally at a dose of 75 mg/m 2.

16. The method of any one of claims 1-4, wherein the tamibarotene is administered orally at a dose of 6 mg twice per day on each of days 8-28 of a 28-day treatment cycle and azacitidine is administered intravenously or subcutaneously at a dose of 75 mg/m 2 on each of days 1-7 of the 28-day treatment cycle.

17. The method of any one of claims 1-4, wherein the method comprises administering a second therapeutic agent and a third therapeutic agent, wherein the second therapeutic agent and the third therapeutic agent are independently selected from an HMA, optionally azacitidine or decitabine, a BCL2 inhibitor, optionally venetoclax, low-dose ara-C(LDAC), obinutuzumab, rituximab, a CD47 inhibitor, optionally magrolimab, and an androgen.

18. The method of any one of claims 1-4, wherein the method comprises administering a second, a third, and a fourth therapeutic agent, wherein the second, third, and fourth therapeutic agents are independently selected from an HMA, optionally azacitidine or decitabine, a BCL2 inhibitor, optionally venetoclax, low-dose ara-C(LDAC), obinutuzumab, rituximab, a CD47 inhibitor, optionally magrolimab, and an androgen.

19. The method of claim 18, wherein
the second agent is an HMA, optionally azacitidine or decitabine, the third agent is a BCL2 inhibitor, optionally venetoclax, and the fourth agent is LDAC;
the second agent is an HMA, optionally azacitidine or decitabine, the third agent is a BCL2 inhibitor, optionally venetoclax, and the fourth agent is obinutuzumab;
the second agent is an HMA, optionally azacitidine or decitabine, the third agent is a BCL2 inhibitor, optionally venetoclax, and the fourth agent is rituximab;
the second agent is an HMA, optionally azacitidine or decitabine, the third agent is a BCL2 inhibitor, optionally venetoclax, and the fourth agent is a CD47 inhibitor, optionally magrolimab;
or
the second agent is an HMA, optionally azacitidine or decitabine, the third agent is a BCL2 inhibitor, optionally venetoclax, and the fourth agent is an androgen.

20. The method of claim 19, wherein the method comprises
administering an HMA, optionally azacitidine or decitabine, as the second agent, a BCL2 inhibitor, optionally venetoclax, as the third agent, and LDAC, as the fourth agent to a patient with AML, optionally the M4 or M5 subtype of AML;
administering an HMA, optionally azacitidine or decitabine, as the second agent, a BCL2 inhibitor, optionally venetoclax, as the third agent, and obinutuzumab as the fourth agent to a patient with CLL or SLL;
administering an HMA, optionally azacitidine or decitabine, as the second agent, a BCL2 inhibitor, optionally venetoclax, as the third agent, and rituximab as the fourth agent to a patient with CLL or SLL, with or without 17p deletion;
administering an HMA, optionally azacitidine or decitabine, as the second agent, a BCL2 inhibitor, optionally venetoclax, as the third agent, and a CD47 inhibitor, optionally magrolimab, as the fourth agent to a patient with an MDS; or
administering an HMA, optionally azacitidine or decitabine, as the second agent, a BCL2 inhibitor, optionally venetoclax, as the third agent, and an androgen, as the fourth agent to a patient with persistent anemia.

21. A method of treating a population of patients who each have a hematopoietic cancer, the method comprising administering to the patients about 12 mg of tamibarotene per day, wherein the about 12 mg of tamibarotene is administered regardless of the patients' weight or body surface area.

22. The method of claim 21, wherein the about 12 mg of tamibarotene is divided into two doses, the first being a first daily dose and the second being a second daily dose.

23. The method of claim 22, wherein the first daily dose and the second daily dose each contain about 6 mg of tamibarotene.

24. The method of claim 21, wherein the tamibarotene is administered orally.

25. The method of any one of claims 21-24, wherein the population of patients comprise human children, adolescents, and/or adults.

26. The method of any one of claims 21-24, wherein the population of patients comprises adult humans.

27. The method of any one of claims 21-24, wherein the hematopoietic cancer is a leukemia, optionally a leukemia resistant or refractory to treatment with venetoclax, a lymphoma, multiple myeloma, or myeloid neoplasm with myelodysplasia.

28. The method of claim 27, wherein the leukemia is acute lymphoblastic leukemia (ALL), chronic lymphocytic leukemia (CLL), acute myeloid leukemia (AML), or chronic myeloid leukemia (CML), and the lymphoma is Hodgkin lymphoma or non-Hodgkin lymphoma.

29. The method of claim 28, wherein the AML is non-acute promyelocytic leukemia acute myeloid leukemia (non-APL AML).

30. The method of claim 27, wherein the myeloid neoplasm with myelodysplasia is a myelodysplastic syndrome (MDS).

31. The method of claim 30, wherein the MDS is higher risk MDS.

32. The method of any one of claims 21-24, wherein the method comprises administering a second therapeutic agent, wherein the second therapeutic agent is a hypomethylating agent (HMA) or a BCL2 inhibitor.

33. The method of claim 32, wherein the HMA is decitabine or azacitidine and the BCL2 inhibitor is venetoclax.

34. The method of claim 33, wherein the HMA is azacitidine.

35. The method of claim 34, wherein the azacitidine is administered parenterally at a dose of 75 mg/m 2.

36. The method of any one of claims 21-24, wherein the tamibarotene is administered orally at a dose of 6 mg twice per day on each of days 8-28 of a 28-day treatment cycle and azacitidine is administered intravenously or subcutaneously at a dose of 75 mg/m 2 on each of days 1-7 of the 28-day treatment cycle.

37. The method of any one of claims 21-24, wherein the method comprises administering a second therapeutic agent and a third therapeutic agent, wherein the second therapeutic agent and the third therapeutic agent are independently selected from an HMA, optionally azacitidine or decitabine, a BCL2 inhibitor, optionally venetoclax, low-dose ara-C(LDAC), obinutuzumab, rituximab, a CD47 inhibitor, optionally magrolimab, and an androgen.

38. The method of any one of claims 21-24, wherein the method comprises administering a second, a third, and a fourth therapeutic agent, wherein the second, third, and fourth therapeutic agents are independently selected from an HMA, optionally azacitidine or decitabine, a BCL2 inhibitor, optionally venetoclax, low-dose ara-C(LDAC), obinutuzumab, rituximab, a CD47 inhibitor, optionally magrolimab, and an androgen.

39. The method of claim 38, wherein
the second agent is an HMA, optionally azacitidine or decitabine, the third agent is a BCL2 inhibitor, optionally venetoclax, and the fourth agent is LDAC;
the second agent is an HMA, optionally azacitidine or decitabine, the third agent is a BCL2 inhibitor, optionally venetoclax, and the fourth agent is obinutuzumab;
the second agent is an HMA, optionally azacitidine or decitabine, the third agent is a BCL2 inhibitor, optionally venetoclax, and the fourth agent is rituximab;
the second agent is an HMA, optionally azacitidine or decitabine, the third agent is a BCL2 inhibitor, optionally venetoclax, and the fourth agent is a CD47 inhibitor, optionally magrolimab; or
the second agent is an HMA, optionally azacitidine or decitabine, the third agent is a BCL2 inhibitor, optionally venetoclax, and the fourth agent is an androgen.

40. The method of claim 39, wherein the method comprises
administering an HMA, optionally azacitidine or decitabine, as the second agent, a BCL2 inhibitor, optionally venetoclax, as the third agent, and LDAC, as the fourth agent to a patient with AML, optionally the M4 or M5 subtype of AML;
administering an HMA, optionally azacitidine or decitabine, as the second agent, a BCL2 inhibitor, optionally venetoclax, as the third agent, and obinutuzumab as the fourth agent to a patient with CLL or SLL;
administering an HMA, optionally azacitidine or decitabine, as the second agent, a BCL2 inhibitor, optionally venetoclax, as the third agent, and rituximab as the fourth agent to a patient with CLL or SLL, with or without 17p deletion;
administering an HMA, optionally azacitidine or decitabine, as the second agent, a BCL2 inhibitor, optionally venetoclax, as the third agent, and a CD47 inhibitor, optionally magrolimab, as the fourth agent to a patient with an MDS; or
administering an HMA, optionally azacitidine or decitabine, as the second agent, a BCL2 inhibitor, optionally venetoclax, as the third agent, and an androgen, as the fourth agent to a patient with persistent anemia.
